(19) United States
(12) Patent Application Publication
(10) Pub. No.: US 2022/0023468 A1
(43) Pub. Date: Jan. 27, 2022

(54) CONTROL SURFACES AND SYSTEMS AND METHODS FOR SANITATION OF CONTROL SURFACES

(71) Applicant: Bruce Aerospace Inc., Dayton, NV (US)

(72) Inventor: Storm S. Sears, Dayton, NV (US)

(21) Appl. No.: 17/382,430

(22) Filed: Jul. 22, 2021

Related U.S. Application Data
(60) Provisional application No. 63/055,557, filed on Jul. 23, 2020.

(51) Int. Cl. A61L 2/10; A61L 2/24

(52) U.S. Cl. CPC A61L 2/10; A61L 2/24; B64F 5/30; A61L 2202/14; A61L 2202/11

(57) ABSTRACT

The invention relates to sanitizing surfaces designed to be touched with sanitation systems and methods for sanitizing such surfaces, such as a control surface which must be touched to either directly or indirectly cause an action. At least one irradiation system includes at least one UVGI emitter in at least one array configured to irradiate at least a portion of a predetermined surface with a predetermined dose of UV radiation, such as after touching of the surface.

### CROSS REFERENCE TO RELATED APPLICATIONS

This application claims benefit of pending U.S. Provisional Patent Application No. 63/055,557, filed Jul. 23, 2020, the contents of which is incorporated herein by reference.

### TECHNICAL FIELD

The invention relates to sanitizing predetermined surfaces, such as control and/or action surfaces, high touch surfaces or the like, with sanitizing systems for use with such predetermined surfaces and methods for sanitizing such predetermined surfaces. For example, a surface designed to be touched may be a control surface, being a surface on an item or structure which is designed to be touched, or an action surface being a surface on an item or structure which is touched to directly or indirectly cause an action.

### BACKGROUND OF THE INVENTION

The spread of disease by contact with surfaces that are often touched by others, has been an issue for some time, and has become more prevalent with increased presence of new dangerous pathogens being encountered in the environment, and transmitted between people. There are generally two methods of infectious disease propagation which are of primary concern, being airborne transmission of pathogen bearing droplets from an infected individual to healthy individuals, and the physical transmission of pathogens, such as viral microbes or bacteria, arising from contact with contaminated surfaces touched by infected individuals. In the public or other environments, there are many surfaces designed to be touched, such as control surfaces and/or action surfaces that require touching by an individual, such as to carry out some function. Such control surfaces may be mechanical in nature, such as latches, handles, valves, knobs or the like. The control and/or action surfaces may also be electronic or electro-mechanical in nature such as buttons, switches, keypads, touch control surfaces, slides or a myriad of other devices.

The traditional method for surface disinfection involves physically cleaning surfaces with a combination of surfactants and disinfectants, by spraying or rubbing for example. A problem relating to the disinfection using aerosol or aqueous cleaning and disinfection products is that such products can damage control surfaces, particularly electronic control surfaces. Further, the surface may need to remain moistened for a period of time for the disinfectant to adequately deactivate a suitable percentage of the population of pathogens present on the surface, to satisfactorily prevent the pathogens from infecting other people who touch the control surface. Such a process requires the close attention of the person cleaning the surfaces to ensure that the surfaces remain wet with the disinfectant for at least the minimum length of time mandated by the specific product being used. Another method involves the large-scale exposure of surfaces to UV radiation in the germicidal wavelength range (200-280 nm). Either of these disinfection methods; however, are limited in effectiveness with regard to control surfaces which have a high probability of direct and recurring contact, ensuing contamination, and resulting disease transmission.

These common disinfection methods are inadequate to keep a surface disinfected in between each touch or use, and do not adequately focus on surfaces designed to be touched. As a result, these surfaces may not receive adequate disinfection during regular cleaning cycles. In addition, control surfaces may be complex in shape, or of a recessed or hidden design, which makes them difficult to clean and disinfect. For example, such complex shapes make it difficult to wet all of the various contours, joints, or other features of the control surface, and other hard-to-reach places with a chemical disinfectant. Such complex shapes and configurations also may inhibit irradiation using a broad beam UVC source. Also, as such UVC radiation is damaging to human tissue and cells, the use of UVC radiation and disinfection of an area while occupied is potentially dangerous.

In different environments, there are surfaces designed to be touched, which may include control and/or action surfaces and also high touch surfaces, which present ideal surfaces for transmission of pathogens, as it has not been possible to disinfect such surfaces between touches when the chance of multiple people touching such surfaces in a short amount of time is likely. Such environments include commercial transportation vehicles, hospitals, commercial or retail environments and many other like public or private environments. In commercial transportation vehicles for example, high touch surfaces may include tray tables and latches; premium aircraft seat pocket latches; lavatory door latches, lavatory faucet and toilet controls; aircraft cabin overhead PSU controls, overhead storage bin latches, arm rests and the like. In a hospital or healthcare environment for example, such high touch surfaces may include bed rails, bed frames, lights, tray or bedside table, handles, IV poles or other medical equipment and many more. In a commercial or retail type of environment for example, such high touch surfaces may include door handles, shopping carts, payment systems, seating and/or tables or furniture, elevator controls or a wide variety of other similar surfaces. It would be desirable to be able to disinfect such surfaces more effectively.

It would thus be highly desirable to prevent subsequent spread of pathogens transferred to such predetermined surfaces, such as control surfaces, action surfaces, high touch surfaces or the like, by properly cleaning and disinfecting between each touching of the predetermined surface. There is a continued need for effective systems and methods to disinfect surfaces and inhibit the transmission of pathogens between people.

### SUMMARY OF THE INVENTION

In one aspect of the present invention, there is provided a predetermined surface, being a surface designed to be touched such as a control surface, action surface or high touch surface, with at least one sanitizing assembly including at least one irradiation system positioned in predetermined relationship to at least a portion of the predetermined surface. The at least one irradiation system includes at least one UVGI emitter in at least one array configured to irradiate at least a portion of the predetermined surface with UV radiation of a predetermined wavelength or range of wavelengths, and at a predetermined energy level. A control system is provided to operate the at least array for an amount of exposure time.

The invention is also directed to a sanitizing system for a predetermined surface, being a surface designed to be touched such as a control surface, action surface, high touch surface or the like, configured for sanitizing a predetermined surface. The sanitizing system includes at least one irradiation system configured to be positioned in predetermined relationship to at least a portion of the predetermined surface, such as by a mounting system. The at least one irradiation system includes at least one UVGI emitter in at least one array configured to irradiate at least a portion of the predetermined control surface with UV radiation of a predetermined wavelength or range of wavelengths, and at a predetermined energy level, when positioned in the predetermined relationship with the predetermined control surface. The sanitizing system includes a control system provided to operate the at least array for an amount of exposure time.

The invention also relates to a method of sanitizing a predetermined surface, being a surface designed to be touched such as control surfaces, action surfaces, high touch surfaces or the like, by providing at least one irradiation system including at least one UVGI emitter in at least one array positioned in predetermined relationship with the predetermined surface. The touching or use of the predetermined surface is detected, and operation of the at least one irradiation system controlled to irradiate at least a portion of the predetermined surface with UV radiation of a predetermined wavelength or range of wavelengths, and at a predetermined energy level and for an exposure time after touching or use of the predetermined surface.

These and other aspects of the invention will become apparent based upon the following description of examples of the invention in conjunction with the figures.

### BRIEF DESCRIPTION OF THE DRAWINGS

FIG. 1 is a somewhat schematic view of a first example of the invention showing a control surface with a disinfecting system incorporated into the control surface, including an irradiation system to disinfect the control surface;

FIG. 2 shows a side view of the system of FIG. 1, according to the example of the invention;

FIG. 3 shows a schematic of a control system for the irradiation system according to an example;

FIG. 4 shows a diagram showing operation of an irradiation system according to an example of the invention;

FIG. 5 shows another example of the invention for a retrofit irradiation system for use with a control surface;

FIGS. 6 and 7 schematically represent another type of control surface with an irradiation system for treatment of at least a portion of the control surface with which it is used;

FIG. 8 schematically represents another type of control surface with an irradiation system for treatment of at least a portion of the control surface with which it is used; and

FIG. 9 shows a flow diagram of an example method according to the invention.

### DETAILED DESCRIPTION OF THE INVENTION

The present invention provides disinfecting systems and methods for predetermined surfaces designed to be touched, such as control surfaces, action surfaces, high touch surfaces or the like. The systems and methods enable more effective decontamination of such surfaces from pathogens including viruses, bacteria or other microorganisms. As an example, the present invention may be used to provide disinfection of any predetermined surface immediately after touching or use of the surface by a person, to ensure proper disinfection between touches or uses of the surface. A surface designed to be touched for example may be a control surface, which is a surface which must be touched for a purpose, such as an action surface to either directly or indirectly cause an action. The touching of the surface may be by a person or may be indirectly touched by another article. It should be recognized that such surfaces designed to be touched can be of a wide variety of shapes and configurations, and may include mechanical objects such as latches, handles, knobs, valves or the like. The surfaces may also be control or action surfaces of an electronic or electro-mechanical nature, such as buttons, switches, keypads, touch control surfaces, slides or a myriad of other devices. Such control and/or action surfaces must be touched to cause an action, and are found in numerous products, objects and environments. Surfaces designed to be touched such as latches, handles, knobs, levers and the like are found in association with doors, hatches, bins, dispensers or a myriad of other objects used in commercial products, public environments, commercial environments, passenger vehicles and the like, as well as a multitude of possible other public and private environments and applications. Control and/or action surfaces such as buttons, switches, keypads, touch control surfaces and the like are used ubiquitously in numerous products, objects and environments, such as association with commercial passenger vehicles, door locks or access panels, ATM's, gas pumps, grocery store checkouts many others. All such surfaces may be contaminated by a person's touch or by the touch of another article that has pathogens on it, and the systems and methods of the invention are directed at sanitizing the predetermined surface immediately after being touched/used, to prevent the transmission of contaminants or disease by pathogens left on the control surface after touching or use. The present systems and methods are thus configured to kill bacteria or inactivate the viral or other pathogen particles on the surface between touching or uses of the surface as an example.

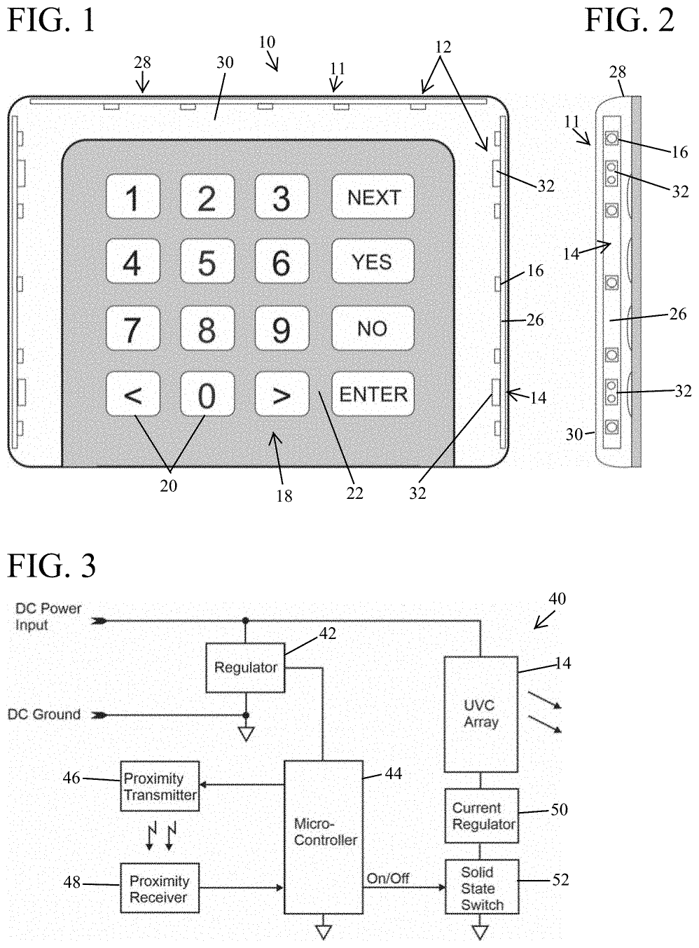

In a first example of the invention as shown in FIGS. 1 and 2, a surface designed to be touched is a control surface 10, used in a wide variety of products and environments and applications. The control surface 10 is a keypad 18 that is designed with a sanitizing system according to the invention integrated into the control surface 10 during manufacture. This example control surface 10 has a keypad 18 with keys 20 that are pushed or touched to input information. The surface of keypad 18, and/or of keys 20 form action surfaces that must be touched to cause an action. These types of control surfaces 10 are used in many different environments and applications, as should be recognized. For example, in a commercial passenger vehicle, such as an aircraft or the like, example applications may include, but are not limited to aircraft cockpit instrument panels; including switches, keypads and touch panels; aircraft passenger cabin flight crew electronic controls; aircraft passenger cabin seat electronic controls, and the like. Similar control surfaces are used in many other public, commercial, private or like environments and applications, such as ATM machine controls; point of sale credit card/ATM card readers; data entry pads; vending machine keypads; elevator keypads; access control panels, and in association with many other objects or environments.

In this example, the predetermined surface designed to be touched is a control surface 10 that includes a sanitizing system with at least one irradiation system 12 including at least one array 14 of one or more radiation sources 16. The at least one array 14 is provided in predetermined relationship to the configuration of the control surface 10, and action surfaces thereof for example. In this example, the integration of the irradiation system 12 into the control surface 10 configuration allows the irradiation system 12 to match the configuration of the control surface 10 to provide proper irradiation and sanitation of the control surface 10 including at least the action surfaces. In this example, the control surface includes a low-profile, U-shaped housing assembly 11 with a UVGI LED array 14 pointing across the keypad 18 surfaces from the sides and top. The at least one array 14 is positioned in predetermined proximity to the at least the action surfaces such as keypad 14 and keys 16 of control surface 10. The radiation sources 16 may be UV LED's, or possibly a fluorescent bulb, excimer lamp or other suitable source, that emits ultraviolet germicidal irradiation (UVGI) such as UVC and/or far-UVC radiation onto the surfaces of the control surface 10. The sources 16 may produce radiation in the germicidal wavelengths of between 200-280 nm for example, which is effective at deactivating pathogens by direct exposure to the UVGI of a predetermined energy level for a predetermined time. The sources 16 may produce radiation in the far-UVC germicidal wavelength of approximately 222 nm for example, which also appears to have less adverse effects on people. The location of the radiation sources 16 are selected to ensure direct exposure of all action or other surfaces associated with the control surface 10, including the tops and sides of keys 20, and the surfaces 22 around the keys 20. As seen in the FIGS., the control surface 10 is somewhat planar and the radiation sources 16 are positioned adjacent at least the top and sides of the control surface 10 to irradiate each of the keys 20 and surfaces 22 over the entire control surface 10. This placement of the arrays 14 effectively treats the entire control surface 10 as well as each of the keys 20. The internal surface of the housing or bezel assembly 28 may be lined or painted with a UVC reflective material which optimizes the UVC radiation cast on the control surface 10. The geometry of the bezel housing can also be adjusted to optimize the UVC radiation pattern for any predetermined control surface.

This placement of the at least one irradiation array also provides the ability to effectively irradiate the control surface with UVGI in the dosage required, which is based on the distance, intensity, and duration of exposure, to effectively inactivate or kill pathogens on at least a portion of the control surface 10. Generally, the UVGI dosage amounts decrease significantly by approximately the square of the distance from the radiation source, so the placement directly adjacent the control surface 10 avoids significant attenuation of the UV irradiation. The intensity or energy output of the UVGI radiation sources 16 and position and distance from the surfaces of control surface 10 are chosen to provide the desired efficacy in inactivating or killing any pathogens in association with a predetermined control surface, in a predetermined amount of time. In many applications and environments, the arrays 14 may position the UV radiation sources 16 within approximately 5 cm of the surfaces of control surface 10 to be irradiated, to reduce the energy required from the UVGI radiation sources 16 for example. But, depending on the control surface, the sources 16 and exposure time of irradiation may be chosen based on the need to be further from the control surface, to provide the desired predetermined dosage to the control surface. The UVGI intensity amount required to kill most bacteria and inactivate viruses is between 2,000-8,000 μW·s/cm², and the arrays 14 may provide the desired dosage to every portion of the control surface 10. To accurately provide the desired UV dose, the UV intensity, adjusted for distance, coating, and end of lamp life, will be multiplied by the exposure time. Depending on the nature of the control surface 10, it may be expected to be touched/used often, or more intermittently, and the UVGI dose can be selected in accordance with the particular type of control surface and environment in which it is used to provide the effective UV dose to be reached in a predetermined amount of time. The ability to tailor the irradiation system to the control surface enables effective sanitizing in various environments and applications. In addition, depending the type and environment in which a control surface 10 is used, the irradiation system 12 may be configured to account for the possibility that the microorganisms are protected by mechanical particles, such as dust and dirt, or have formed a biofilm, which may require a higher UV fluence for an effective UV dose to be introduced to the microbes.

For people, it is known that skin exposure to at least certain UVGI wavelengths of UVC light can produce rapid sunburn and even result in skin cancer. It is also known that exposure of the eyes to this UVC radiation can damage the cornea or retina. Such UVC radiation can also cause the production of ozone, which can be harmful to people. The irradiation system 12 thus can be desirably configured to minimize any exposure of the skin or eyes to the UVGI radiation produced by the arrays 14, and to minimize the production of ozone if desired. In this example, the face of the control surface 10 remains open and accessible at all times. This means that when the UVGI array 14 is actuated, there may be some minor amount of UVC radiation that is reflected away from the control surface 10 into potentially inhabited space. However, due to the fact that a low-power UVC LED array may be employed and the active time of the LED array is brief as described below, this minor amount of open air UVC radiation poses no health risk to nearby individuals. In addition, the primary radiation of the UVGI LED array is focused on the control surface, not randomly into space, for the purpose of achieving the highest sanitizing efficiency. The proximity of the sources 16 to the control surface 10 allows lower energy sources 16 to be used, and the dose of UVGI that can result in the vicinity of the control surface 10 is small because of attenuation. But, if desired, the control surface 10 may include shielding positioned between the sources 16 and a user, to minimize any radiation exposure in the vicinity of the control surface 10. But, the system may even allow for additional application as the actuation of the irradiation system is brief, and some UVGI wavelengths may be safer for people to be exposed to. For example, the control surface 10 could allow for sanitation of a user's hands or fingers, such as by allowing the user to select this operation and maintain their hands or fingers in the zone of irradiation for the control surface 10 during an exposure period.

In this example, the sources 16 are mounted in conjunction with a printed circuit board (PCB) 26, and may be positioned in a housing 28, with sides 30 extending above and toward the control surface 10 to provide a barrier between the sources 16 and the user. The internal surface of the housing 28 may be lined or painted with a UVC reflective material which optimizes the UVC radiation cast on the keypad 18 of control surface 10. Reflectance of the UVGI radiation by the housing 28 onto the control surface 10 may also enhance disinfection performance by ensuring all surfaces are exposed to direct UVGI radiation. The geometry of the bezel housing 28 can also be adjusted to optimize the UVC radiation pattern. In this example, it is presumed that the keypad control surface 10 is mounted below line-of-sight, in other words, below the height of most people's heads. The housing 28 is configured without an array assembly 14 mounted on the bottom side of the keypad 18. In this way, inadvertent direct viewing of an array assembly 14 is avoided while it is activated. In other applications, the at least one housing 28 could take a variety of shapes in order to best irradiate the control surface and to also prevent direct viewing of the UVGI array when active if needed. The housing 28 may be constructed to prevent degradation of the material from which it may be constructed by exposure to the UVGI radiation.

As seen in FIGS. 1-2, the UVGI LED boards 26 in this example, may be linear to extend the entire length of the housing 28 adjacent the control surface 10, but the array 14 may be of any desired configuration. There may also be provided heat-sinking for the sources 16 and boards 26 to keep the UVGI emitters 16 at an optimal operating temperature. Further, the sources 16 may be configured to emit UVC or far-UV radiation in specific wavelengths for optimized performance in the environment in which the control surface 10 is used, such as in aircraft or other commercial passenger vehicles or many other applications and environments. For example, the sources 16 may be configured to emit UVGI in the energy range of about 3.5-10 eV at the location of the control surface to provide high-energy photons to cleave most chemical bonds and kill microbes by destroying nucleic acids and disrupting their DNA, or inactivating viruses. The LED board assemblies 26 may also incorporate the current regulation circuitry for the UV emitters 16 to keep them operating at a precise operating current level over temperature. The LED's 16 may incorporate a lens assembly formed from special UV transmissive silicone rubber, in order to protect the UV LED emitters 16 from physical damage.

The UVGI array assembly 14 may be powered from available power where the control surface is used, or the control surface 10 may include a battery or other suitable power source. The UVGI array assembly 14 electronics may also incorporate at least one sensor 32, such as a proximity or motion sensor for example, to turn on the array assembly 14 after use of the control surface 10, for a predetermined exposure time. An aspect of the invention is that the control surface 10 is only irradiated long enough to ensure that the surface is fully disinfected or sanitized, which for many types of control surfaces 10 may only amount to a period of a few seconds. The use of a proximity, motion or other suitable sensor 32 with the array system 14 reduces the possible exposure to UVGI radiation and minimizes power requirements for operation of the system 14. The at least one sensor 32 may be for example, visible light or IR beam interruption sensor system, positioned on opposing upper and lower sides of the keypad 18, to sense the approach of fingers or other object. When a hand or other object comes into close proximity to the control surface 10, this presence is sensed by sensor(s) 32. When the hand or other object leaves the control surface area, the UVGI array 14 is actuated for a brief interval or exposure period sufficient to sanitize the control surface 10. Any suitable sensor 32 arrangement may be used, to detect movement/presence of objects without physical contact, and relay that information captured into an electrical signal. Such sensors may include a beam interruption system, motion detector, IR, ultrasonic, microwave or other proximity sensors, photoelectric sensors, passive IR (PIR), or any other suitable method or device. In the case of visible light beam or IR beam interruption sensors, the projected beam will be modulated in a particular manner so that it can be discriminated from background ambient light or environmental IR radiation upon reception, to prevent unwanted actuation or deactivation of the array assembly 14.

As seen in FIG. 3, a control system 40 for the UVGI array 14 may include a voltage regulator 42 to provide conditioned power to a microcontroller 44. Connected to the microcontroller 44 is a proximity sensor, such as a proximity transmitter 46 and proximity receiver 48. Upon detection of a hand or other object leaving the control surface area by the proximity sensor, the UVGI array 14 is actuated by microcontroller 44 for a brief interval in conjunction with a current regulator 50 and switch 52, being an interval predetermined for the control surface 10 which is sufficient to sanitize the control surface 10.

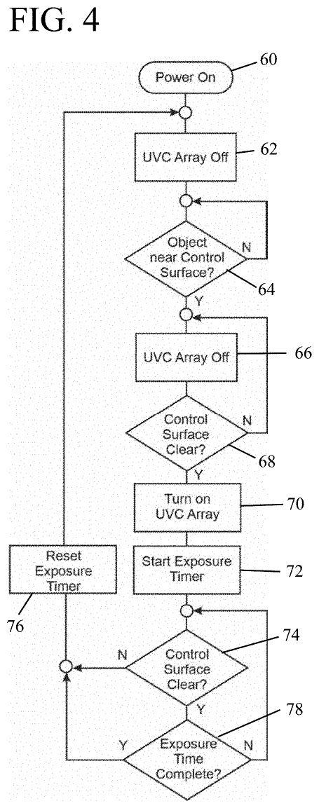

An example of the operation of the array system 14 is seen in FIG. 4, which includes providing power at 60 and using the switch 52 to turn the irradiation system off at 62. The control system 40 logic associated with microcontroller 44 determines if an object is sensed near the control surface at 64 via a signal from sensor 32, and if not maintains the array system 14 powered off. If an object is sensed near the control surface at 64, the array is maintained off at 66 and a determination is made as to whether the object has been removed such that the control surface is clear at 68. If not, the array is maintained powered off, but upon clearing of the control surface as determined at 68, the array is then turned on at 70, and an exposure timer started at 72. It is then determined if the control surface remains clear at 74, and if not, the exposure timer is reset at 76, the operation restarted at 62 and array 14 is turned off. If the control surface remains clear at 74, it is then determined if the exposure time was completed at 78, and if not, the determination of whether the control surface is clear is repeated at 74 until the exposure time is completed at 78, at which point the exposure timer is reset at 76, and the operation restarted at 62.

The array 14 is thus activated for a predetermined amount of time after use of the control surface as detected by an object coming into proximity with the control surface 10. If another object comes into proximity during the exposure period, the array 14 may be turned off to prevent exposure of another user to the radiation, or because the exposure level may still be minimal, other control of the system may be performed to ensure complete sanitization of the control surface 10. If the system is prematurely turned off before the full exposure period is reached, a warning or the like may be issued to alert the next user to this situation if desired. Alternatively, a barrier may be provided and moved to prevent use of the control surface 10 for the exposure period, and then removed to allow use of the control surface 10. The control surface 10 may further include an indicator or other suitable system to let any persons in the area of the control surface 10, that the irradiation system 12 is in operation. Other suitable arrangement to facilitate avoiding use of the control surface 10 before the desired exposure time is complete after a use are contemplated.

The low-power irradiation array system 14 provides a simple, low-profile assembly, and is integrated directly into the design of the control surface 10 itself. This allows the system 14 to be specifically configured for a particular control surface, and as noted, such control surfaces can vary significantly. In the example of FIGS. 1 and 2, the system 14 is relatively simple in that the control surface 10 is a planar keypad, but would be configurable to irradiate all surfaces of a control surface of any other configuration.

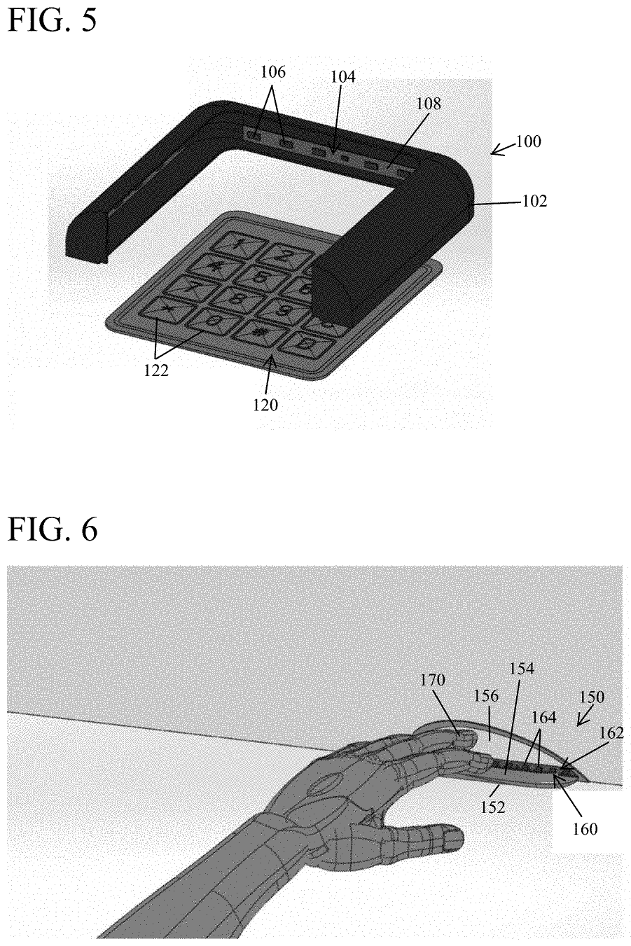

Turning to FIG. 5, another example of the invention relates to providing a sanitation system 100 including an irradiation system, that is retrofit for use with an existing surface designed to be touched, such as a control surface 120. Again in this example, the control surface 120 is a keypad, with keys 122 that are touched or pushed by the user to affect an action, such as to make a selection, input information or the like. The keypad 120 could be part of a myriad of devices/objects, such as noted with respect to the example of FIGS. 1 and 2, or in association with any other objects or environments. As the control surface 120 does not have any way to disinfect the surfaces of the control surface 120 associated therewith, a separate irradiation system 100 is configured to be mounted in association with the control surface 120. In this example, the configuration of irradiation system 100 may be similar to the integrated system 14 of FIGS. 1 and 2, as the control surface 120 is a relatively simple planar surface with keys 122. The irradiation system 100 may thus be retrofit to the control surface 120, but configured to properly disinfect the control surface 120 as desired. The sanitizing irradiation system 100 thus may include a housing 102 with at least one irradiation array 104, with UVGI sources 106 mounted in conjunction with a printed circuit board (PCB) board 108 as in the prior example, or any suitable arrangement. The irradiation system 100 may operate in a manner similar to that described with the integrated irradiation system of FIGS. 1 and 2 for example, and include at least one sensor to activate the system after use of the control surface 120. This allows existing control surfaces 120 to be disinfected in an effective manner to prevent the transmission of contaminants or disease as desired, by ensuring all surfaces of the control surface 120 are exposed to direct UVGI radiation.

The invention as described can be applied to any control surface, either by integration of a sanitizing system including a UVGI irradiation system tailored for the particular control surface, or as a retrofit irradiation system designed for existing surfaces designed to be touched, such as control surfaces that are in use. Various control surfaces are configured in a manner that also generally result in interaction with only a portion of the control surface. For example, with respect to keyboards, the keys are pushed or touched and the other surfaces of the control surface are not required to be pushed or touched, and the irradiation system of the invention may be configured to irradiate the portions of a control surface that are primarily susceptible to contamination and/or action surfaces. For example, a control surface such as a door handle may generally include portions designed to be grasped by a person, while other portions are generally not touched. The irradiation system could be configured to disinfect the portions designed to be grasped or touched by a person if desired.

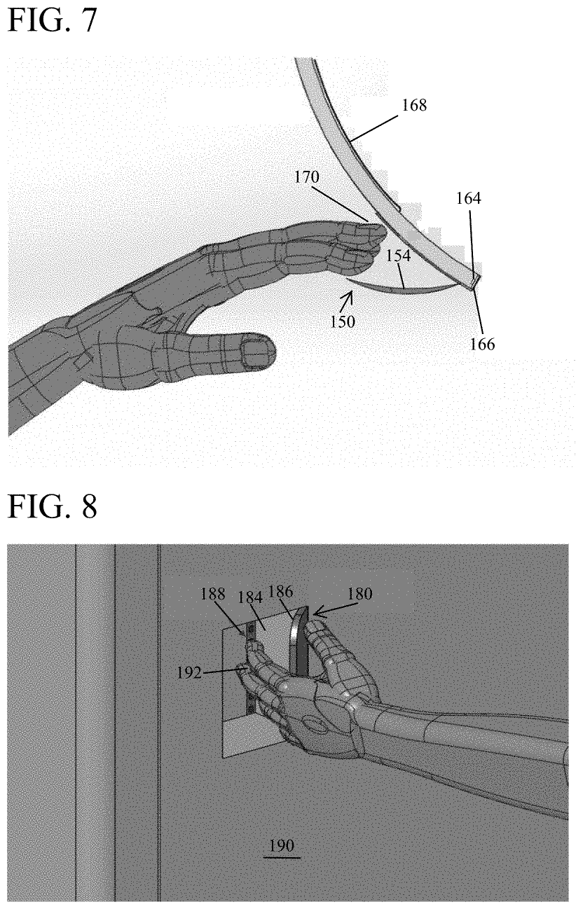

Turning to FIGS. 6 and 7, another example of a control surface that it is desired to disinfect to prevent infection or contamination to users of the control surface. In this example, the control surface 150 is a recessed latch 152, such as may be found in association with overhead stow-bin latches aboard commercial aircraft or the like. The system of the invention may be integrated into the latch 152 or provided as a retrofit kit for the specific style of latch 152. Recessed latch assemblies are commonly employed on commercial aircraft or like passenger vehicles to prevent potential injury in the event that someone falls against one of these surfaces. However, due to the recessed design of these types of control surfaces, it is difficult to properly sanitize the internal surface of the latch that people must touch in order to actuate the latch. Such a latch control surface 150 has concealed or hidden surfaces 154 that are generally required to be touched to operate the latch. In order to actuate the recessed latch, it is necessary to place the fingers 170 within a recessed area 156, pulling the latch 152 outwards by touching the concealed surface 154. Such control surfaces 150 are difficult to wipe down and disinfect using known techniques, but are still susceptible to contamination. Other techniques, such as large-scale, high-power UVC irradiation sources used to sanitize aircraft cabin surfaces or surfaces in other similar environments and applications, when passengers aren't present, still would not be effective to disinfect these types of control surfaces. In these types of control surfaces 150, or any control surface having concealed or unexposed surfaces, using chemical cleaners or environmental UV irradiation still may not effectively disinfect such areas, but the systems and methods of the invention allow such control surfaces to be effectively disinfected. In this example, an irradiation system 160, being either integrated or retrofit in association with the control surface 150, is positioned adjacent the concealed portions of control surface 150, being the back surface portion 154 and recessed area 156 of the latch 152 that are likely to require touching to operate the latch 152. The irradiation system 160 may include at least one irradiation array 162, with UVGI sources 164 mounted in conjunction with a printed circuit board (PCB) board 166 as in the prior example, or any suitable arrangement. The irradiation system 160 may operate in a manner similar to that described with the prior examples of irradiation systems for example, and include at least one sensor to activate the irradiation system 160 after use of the control surface 150. This allows the concealed portions 154 and 156 of the control surface 150 to be disinfected in an effective manner to prevent the transmission of disease or contamination as desired. Additional arrays 162 may be positioned to irradiate other portions of the control surface 150 as may be desired. Power to the irradiation arrays may be provided in any suitable manner, such as being connected via a wire to the aircraft power for example, or in another suitable manner, such as including a battery power source. In the example shown, a power wire run could be provided on the back side of the stow bin door, and covered with protective channel in order to prevent damage from an overstuffed stow bin. In this application, the UVGI LED array would be powered directly from aircraft 28 Vdc power. As should be recognized, control surfaces such as in this example are found in many different environments and applications. In this example of a commercial passenger vehicle control surface 150, other examples may include tray table latches; premium aircraft seat pocket latches; lavatory door latches, lavatory faucet and toilet controls; aircraft cabin overhead PSU controls and similar control surfaces in many other public, commercial or like environments and applications as should be recognized. The systems could even be used in association with similar control surfaces in the home environment to facilitate maintenance of good health and cleanliness.

In another example as seen in FIG. 8, a surface designed to be touched may be a control surface 180 such as a recessed door latch 182, used to operate a lock or latch associated with the door 190. Such control surfaces may be used in an aircraft lavatory door handle for example, or many other environments or applications. The control surface 180 may include an integrated or retrofit irradiation system. In this example, the control surface 180 again includes a recessed area 184 with a concealed surface 186 that is touched to operate the latch 182. In order to actuate the recessed latch 182, it is necessary to place the fingers 192 within a recessed area 184, pulling the latch 182 outwards by touching the concealed surface 186. Again, such control surfaces 180 are difficult to wipe down and disinfect using known techniques, but are still susceptible to contamination. In this example, an irradiation system 188, being either integrated or retrofit in association with the control surface 180, is positioned adjacent the concealed portions of control surface 180. The irradiation system 188 may again include at least one irradiation array with UVGI sources mounted in conjunction with a printed circuit board (PCB) board or another suitable arrangement. The irradiation system 188 may operate in a manner similar to that described with the prior examples of irradiation systems for example, and include at least one sensor to activate the irradiation system 188 after use of the control surface 180. This allows the concealed portions 184 and 186 of the control surface 180 to be disinfected in an effective manner to prevent the transmission of disease or contamination as desired. Additional arrays may be positioned to irradiate other portions of the control surface 180 as may be desired. Power to the irradiation arrays may be provided in any suitable manner. Again, it should be recognized that surfaces designed to be touched, such as control surfaces as in this example are found in many different environments and applications. It should also be recognized that such control surfaces come in other form factors, such as simply being a handle which is grasped by the person to open the door 190. An irradiation system according to the invention can be provided in association with such as handle just as in the examples described herein, as well as other types of control surfaces, such as valves, knobs or the like.

At this time, types of UVGI sources, such as UVC LED devices, are still an emerging technology, and as such, the cost of these devices is still quite high. Although, this is a rapidly growing sector within the LED industry and the price of these devices will possibly continue to improve, it is unlikely that sources such as UVC LED devices will ever reach a comparably low price point to visible light LED devices. This is due to the fact that UVC germicidal LED devices require costlier materials in their construction. As an example, plastic materials used in visible light LEDs would degrade rapidly under UVC radiation. As a result, UVC LED devices require either quartz glass, or special silicone lens to be used in their construction. Thus, it is currently an expensive proposition to use high-power UVC LED arrays to blanket an area with a sufficient power density of UVC radiation to ensure surface disinfection. But, the systems and methods of the invention instead allow selectively radiating only those surfaces that are likely to be touched in a public venue for example, and by placing the UVC LED array close to this surface, low-cost, low-power LED emitters can be utilized, making the systems cost-effective, and able to be used to disinfect many different control surfaces in many different environments and applications. The systems and methods further allow for the use of lower-cost UVC LED emitters even though they may suffer from limited lifespan in comparison to higher quality UVC LED devices. By only activating the low-power UVC LED array in the systems and methods when necessary and only for a brief time, the usable lifespan of the UVC LED emitters and thus, the sanitizing apparatus is vastly extended. As UVC LED emitters or other UVGI sources are also more dangerous to people, minimizing any exposure is desirable, and achieved by properly disinfecting high touch areas in between each usage, while minimizing operation of the UVGI array. Thus, even if the cost of UVGI emitters becomes is reduced over time, the attributes of the systems and methods of the invention continue to provide advantages. For example, because a low-power UVC LED array is being employed, in an intermittent fashion, the power consumption of the device is quite low. This eases installation by making it easier to power the device. For the same reasons, heat dissipation from the low-power LED array will be quite low, making it possible to maintain a low junction operating temperature for the UVC LED or other devices without any substantial heat sinking being required. This translates directly to a compact form factor for the systems.

Further, as UVC radiation, particularly at high power levels, has a destructive effect not only on organisms and viral particles, it also has an impact on any organic compound. As a result, materials such as plastics, adhesives, cellulose based materials, organic fibers, and pigments, are rapidly degraded by UVC irradiation. This is currently an issue of concern in commercial aircraft passenger cabins where UVC blanket irradiation methods are being attempted to disinfect cabin interiors in the wake of outbreaks of disease caused by pathogens that may linger on surfaces. Attempts at large-scale UVC cabin disinfection may use UVC disinfecting carts which are brought aboard between flights, when passengers are not present. They may employ high-intensity mercury tube UVC sources held on articulating arms which are moved around the cabin. Repeated usage of these UVC disinfecting carts will undoubtedly lead to accelerated wear or fading of materials on all surfaces. This systems and methods of the invention mostly avoid such problems, by only irradiating surfaces that people are likely to touch and only with intermittent and just enough UVC radiation to ensure proper disinfection, limiting potential wear of bulk surfaces. This invention also makes it possible to limit the frequency of large-scale disinfection cycles that may be used in the cabin, extending the life of the surface materials and saving substantial labor and cost.

Likewise, regular cleaning with surfactants and disinfectants can have a damaging effect on surfaces. Control surfaces such as membrane or pushbutton keypads, touch screens, and switches are particularly susceptible to damage or degradation from chemical cleaning and disinfection. As an example, cleaning solutions may cause buttons to stick, control keypad nomenclature/marking to fade or become unreadable, render LCD or LED displays unreadable or other problems. Cleaning with aqueous or aerosol cleaning agents may also result in moisture ingression within components with which an electronic control surface is used, leading to malfunction. The systems and methods of the invention again avoid such problems and deficiencies of other disinfection approaches. The systems and methods of the invention also overcome the deficiency of regular cleaning using chemicals or general UVC blanket irradiation methods that do not account for usage of and possible contamination of control surfaces between cleaning cycles.

Though the low power and/or intermittent operation of the irradiation system as part of the system of the invention limits any adverse exposure of people in the vicinity, appropriate safeguards may be taken to shield the UVGI radiation if desired. In the systems of the invention, the UVGI array is positioned in close proximity to the surfaces of the control surface to be treated for maximum effectiveness, and results in the radiation generally not extending any significant distance from the array. This allows the lowest feasible amount of UVGI radiation to be employed to lower the power consumption of the unit for a cost effective system 10, and also minimizes any potential for damage to skin or eyes as the UVC exposure falls off radically with just a few feet of physical separation from the array, and momentary exposure poses no health risk. Further, the UVGI emitters used in an array may be configured to emit far-UV in the range of 200 to 222 nanometers, which effectively eliminates pathogens, but does not adversely affect people or materials that may be exposed to the radiation. The UVGI emitters may include an optical bandpass filter to emit the desired wavelength of UV radiation, or could be monochromatic or quasimonochromatic to emit UV of a desired wavelength or small range of wavelengths. It should be noted that the above are only examples of the systems and methods of the invention, and the configurations of the control surfaces and associated irradiation system can be modified to correspond to any desired control surface. A control surface that incorporates the UVGI array(s) directly into the design of the control surface provides the advantage in allowing a drop-in replacement for an existing control surface configuration in an originally manufactured product. Alternatively, a retrofit design allows flexibility to use a system in conjunction with a control surface already in use. The form factor of the control surface with integrated UVGI irradiation system or retrofit UVGI irradiation system may be of any suitable configuration to provide disinfecting irradiation where it is desired. The provision of discrete systems to enable effective disinfection of control surfaces provides distinct advantage in controlling the possible spread of disease.

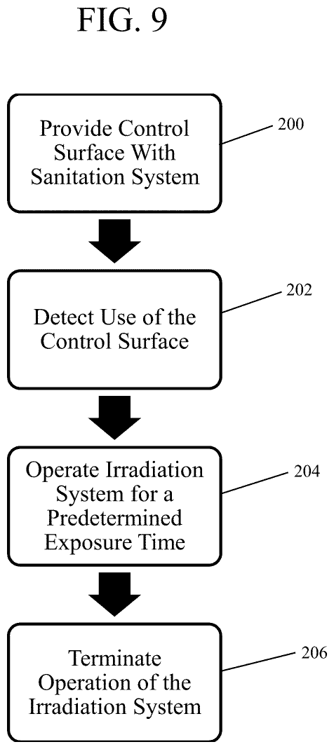

FIG. 9 schematically represents a series of steps involved in a method for treating a surface designed to be touched, such as a control surface or action surface, such as in an aircraft or like commercial passenger vehicle. At 200, a control surface includes at least one sanitation irradiation array with at least one UVGI emitter positioned in predetermined relationship to the control surface. At 202, the presence of a person's touch or the touch of another article to the control surface is sensed. At 204, operation of the at least one UVGI array is initiated to irradiate a predetermined portion of the control surface for a predetermined exposure time. At 206, operation of the at least one array is terminated after irradiation of a predetermined portion of the control surface for a predetermined exposure time.

Although certain examples of the invention have been described, the examples are not limiting and modifications may be made without departing from the spirit and scope of the invention as set forth in the following claims.

What is claimed is:

1. A surface configuration comprising:
at least one predetermined surface being a surface designed to be touched,
the at least one predetermined surface having at least one sanitizing assembly including an irradiation system positioned in predetermined relationship to at least a portion of the at least one predetermined surface,
the at least one irradiation system including at least one UVGI emitter in at least one array configured to irradiate at least a portion of the at least one predetermined surface with UV radiation of a predetermined wavelength or range of wavelengths, and at a predetermined energy level; and
a control system to operate the at least one array for an amount of exposure time.

2. The configuration of claim 1, further comprising at least one sensor to determine if the at least one predetermined surface has been touched, and to provide a control signal to initiate operation of the at least one array after the predetermined surface has been touched.

3. The configuration of claim 1, further comprising at least one sensor to determine if the at least one predetermined surface has been touched and when it is clear, and providing a control signal to initiate operation of the at least one array after the at least one predetermined surface has been touched and after it is clear.

4. The configuration of claim 1, further comprising shielding provided with respect to the at least one array.

5. The configuration of claim 5, wherein the geometry of the at least one array is configured to irradiate the predetermined surface in a predetermined manner.

6. The configuration of claim 1, wherein the surface is a control surface including action surfaces, and the at least one array is configured to irradiate the control surface including action surfaces.

7. The configuration of claim 6, wherein the control surface is mechanical.

8. The configuration of claim 6, wherein the control surface is electro-mechanical or electronic.

9. The configuration of claim 1, wherein the sanitizing assembly is configured to prevent direct irradiation of the eyes of a person touching the predetermined surface.

10. The configuration of claim 1, wherein the at least one array and control system are configured to irradiate the at least one predetermined surface with a predetermined dose of radiation from the at least one array.

11. The configuration of claim 1, wherein the control system is configured to cause the at least one array to be turned off if touching of the predetermined surface occurs during the exposure time.

12. The configuration of claim 1, wherein the at least one array is integrated with the at least one predetermined surface.

13. The configuration of claim 1, wherein the at least one array is retrofit with the at least one predetermined surface.

14. The configuration of claim 1, wherein a system is provided to inhibit touching of the predetermined surface for a predetermined exposure period.

15. The configuration of claim 1, wherein the predetermined surface includes at least one hidden action surface that is touched to either directly or indirectly cause an action, and the at least one array positioned to irradiate the at least one hidden action surface.

16. The configuration of claim 1, wherein the at least one irradiation system is controlled to provide a predetermined dose of UV radiation in a predetermined amount of time after touching of the at least one predetermined surface.

17. A sanitizing system comprising,
at least one irradiation system configured to be positioned in predetermined relationship to at least a portion of a predetermined surface designed to be touched, wherein the at least one irradiation system includes at least one UVGI emitter in at least one array configured to irradiate at least a portion of the predetermined surface with UV radiation of a predetermined wavelength or range of wavelengths, and at a predetermined energy level, when positioned in the predetermined relationship with the predetermined surface, and
a control system to operate the at least array for an amount of exposure time.

18. The sanitizing system of claim 17, wherein the predetermined surface includes at least one hidden surface designed to be touched to either directly or indirectly cause an action, and the at least one array assembly includes an irradiation system positioned in proximity to the at least one hidden surface for irradiation of the at least one hidden surface.

19. The sanitizing system of claim 17, further comprising at least one sensor to determine if the predetermined surface has been touched, and to provide a control signal to initiate operation of the at least one array after the predetermined surface has been touched.

20. A method for sanitizing a control surface comprising:
providing at least one predetermined surface being a surface designed to be touched, the at least one predetermined surface having at least one irradiation system including at least one UVGI emitter in at least one array positioned in predetermined relationship with the at least one predetermined surface, and
controlling the operation of the at least one irradiation system to irradiate at least a portion of the predetermined surface with UV radiation of a predetermined wavelength or range of wavelengths, and at a predetermined energy level and for an exposure time.
